(19) United States
(12) Patent Application Publication
(10) Pub. No.: US 2002/0156023 A1
(43) Pub. Date: Oct. 24, 2002

(54) Lometrexol combination therapy

(75) Inventors: Jacqueline Mary Walling, Bristol (GB); Heather Kay Webb, San Francisco, CA (US); Alan Hilary Calvert, Newcastle (GB); David R. Newell, Northumbria (GB)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Tularik Inc., South San Francisco, CA

(21) Appl. No.: 10/010,740

(22) Filed: Dec. 5, 2001

Related U.S. Application Data
(60) Provisional application No. 60/254,030, filed on Dec. 6, 2000.
(60) Provisional application No. 60/261,134, filed on Jan. 11, 2001.

(51) Int. Cl. A61K 31/7048; A61K 31/704; A61K 38/21; A61K 31/66; A61K 31/525; A61K 31/517

(52) U.S. Cl. 514/27; 424/85.5; 514/34; 514/266.4; 514/110; 514/414; 514/251; 514/449; 424/649; 514/492; 514/521

(57) ABSTRACT

The present invention provides compositions and methods for treating proliferative disorders using combination therapies of lometrexol and other therapeutically active agents. The methods include administration of lometrexol with one or more therapeutically active agents where lometrexol and the therapeutically active agent(s) are delivered in a single composition, where they are administered in separate compositions in a simultaneous manner, where lometrexol is administered first, followed by the therapeutically active agent(s), as well as where the therapeutically active agent(s) is delivered first, followed by lometrexol. In preferred embodiments, the therapeutically active agent(s) has antiproliferative properties.

### CROSS-REFERENCES TO RELATED APPLICATIONS

This application claims the benefit of U.S. Provisional applications Ser. Nos. 60/254,030, filed Dec. 6, 2000 and 60/261,134, filed Jan. 11, 2001, the disclosures of each being incorporated herein by reference.

### STATEMENT AS TO RIGHTS TO INVENTIONS MADE UNDER FEDERALLY SPONSORED RESEARCH AND DEVELOPMENT

Not Applicable.

### BACKGROUND OF THE INVENTION

1. Field of the Invention

The present invention relates to combinations of lometrexol and other therapeutically active agents that are capable of inhibiting abnormal cell proliferation.

2. Background

Cancer is a generic name for a wide range of cellular malignancies characterized by unregulated growth, lack of differentiation, and the ability to invade local tissues and metastasize. These neoplastic malignancies affect, with various degrees of prevalence, every tissue and organ in the body.

Psoriasis, a common chronic skin disease characterized by the presence of dry scales and plaques, is also thought to be the result of abnormal cell proliferation. The disease results from hyperproliferation of the epidermis and incomplete differentiation of keratinocytes. Psoriasis often involves the scalp, elbows, knees, back, buttocks, nails, eyebrows, and genital regions, and may range in severity from mild to extremely debilitating, resulting in psoriatic arthritis, pustular psoriasis, and exfoliative psoriatic dermatitis. No therapeutic cure exists for psoriasis.

Other diseases associated with an abnormally high level of cellular proliferation include rheumatoid arthritis, benign prostatic hyperplasia, restenosis, where vascular smooth muscle cells are involved, inflammatory disease states, where endothelial cells, inflammatory cells and glomerular cells are involved, myocardial infarction, where heart muscle cells are involved, glomerular nephritis, where kidney cells are involved, transplant rejection, where endothelial cells are involved, infectious diseases such as HIV infection and malaria, where certain immune cells and/or other infected cells are involved, and the like. Abnormal cell proliferation is also the primary mechanism mediating diseases in which angiogenesis or neovascularization play a role (e.g., neoplastic diseases, retinopathy, and macular degeneration). Infectious and parasitic agents per se (e.g., bacteria, trypanosomes, fungi, etc.) can also be subject to selective proliferative control.

A multitude of therapeutic agents have been developed over the past few decades for the treatment of various types of cancer. The most commonly used types of anticancer agents include: DNA-alkylating agents (e.g., cyclophosphamide, ifosfamide), antimetabolites (e.g., methotrexate, a folate antagonist, and 5-fluorouracil, a pyrimidine antagonist), microtubule disrupters (e.g., vincristine, vinblastine, paclitaxel), DNA intercalators (e.g., doxorubicin, daunomycin, cisplatin), and hormone therapy (e.g., tamoxifen, flutamide). These agents also have utility as treatments for other proliferative disorders. For example, severe cases of psoriasis may be treated with antiproliferative agents, such as the antimetabolite methotrexate, the DNA synthesis inhibitor hydroxyurea, and the microtubule disrupter colchicine.

The ideal antineoplastic drug would kill cancer cells selectively, with a wide therapeutic index relative to its toxicity towards non-malignant cells. It would also retain its efficacy against malignant cells, even after prolonged exposure to the drug. Unfortunately, none of the current chemotherapies possess an ideal profile. Most possess very narrow therapeutic indexes and, in practically every instance, cancerous cells exposed to slightly sublethal concentrations of a chemotherapeutic agent will develop resistance to such an agent, and quite often cross-resistance to several other antineoplastic agents. Similar limitations apply when these drugs are used as treatments for other proliferative disorders.

In view of the foregoing, there remains a need in the art to provide more efficacious treatment for neoplasia and other proliferative disorders. The concept of combination therapy is well exploited in current medical practice as a method that sometimes results in greater efficacy and diminished side effects relative to the use of the therapeutically relevant dose of each agent alone. In some cases, the efficacy of the drug combination is additive (the efficacy of the combination is approximately equal to the sum of the effects of each drug alone), but in other cases the effect can be synergistic (the efficacy of the combination is greater than the sum of the effects of each drug given alone). This invention fulfills the need for antiproliferative combination therapies that reduce the dosages required for efficacy, thereby decreasing side effects associated with each agent.

### SUMMARY OF THE INVENTION

In one aspect, the present invention provides a composition for the treatment of proliferative disorders, comprising lometrexol or a pharmaceutically acceptable salt thereof and one or more therapeutically effective agents or pharmaceutically acceptable salts thereof.

In certain embodiments, the composition further comprises folic acid.

In certain embodiments, the therapeutically effective agent is an antiproliferative agent. More particularly, in certain embodiments, it is an alkylating drug, an antimetabolite, a microtubule inhibitor, a podophyllotoxin, an antibiotic, a nitrosourea, a hormone therapy, a kinase inhibitor, or an antiangiogenic agent. In further embodiments, it is carboplatin, doxorubicin, gemcitabine HCl, temolozolamide, cyclophosphamide, methotrexate, paclitaxel, etoposide, carmustine, cisplatin, tamoxifen, or interferon.

In a second aspect, the invention provides a method for the treatment of proliferative disorders, comprising administering to a subject in need of such treatment an effective amount of a composition comprising lometrexol or a pharmaceutically acceptable salt thereof and one or more therapeutically effective agents or pharmaceutically acceptable salts thereof.

In certain embodiments, the composition further comprises folic acid.

In certain embodiments, the proliferative disease is cancer. More particularly, a solid tumor (e.g., ovarian, breast, head and neck, prostate, glioma, colon, stomach, hepatic, renal, chondrocytoma, small cell lung carcinoma, non-small cell lung carcinoma, and melanoma), a lymphoma, or a leukemia.

In another embodiment, the proliferative disease is rheumatoid arthritis, psoriasis, or benign prostatic hyperplasia.

In yet another embodiment, the therapeutically effective agent is an antiproliferative agent. More particularly, in certain embodiments, it is an alkylating drug, an antimetabolite, a microtubule inhibitor, a podophyllotoxin, an antibiotic, a nitrosourea, a hormone therapy, a kinase inhibitor, or an antioangiogenic agent. In further embodiments, it is carboplatin, doxorubicin, gemcitabine HCl, temolozolamide, cyclophosphamide, methotrexate, paclitaxel, etoposide, carmustine, cisplatin, tamoxifen, or interferon.

In a third aspect, this invention provides a method for the treatment of proliferative disorders, comprising administering to a subject in need of such treatment an effective first amount of lometrexol or a pharmaceutically acceptable salt thereof and an effective second amount of one or more therapeutically effective agents or pharmaceutically acceptable salts thereof.

In certain embodiments, the one or more therapeutically effective agents comprises folic acid.

In one embodiment, the amount of lometrexol and amount of therapeutically effective agent are administered simultaneously.

In another embodiment, the amount of lometrexol is administered before the amount of therapeutically effective agent. In some embodiments, the lometrexol is administered within one day, one week, or one month of the administration of the therapeutically effective agent.

In yet another embodiment, the amount of therapeutically effective agent is administered before the amount of lometrexol. In some embodiments, the therapeutically effective agent is administered within one day, one week, or one month of the administration of the lometrexol.

In certain other embodiments, the proliferative disease is cancer. More particularly, a solid tumor (e.g. ovarian, breast, head and neck, prostate, glioma, colon, stomach, hepatic, renal, chondrocytoma, small cell lung carcinoma, non-small cell lung carcinoma, and melanoma), a lymphoma, or a leukemia.

In another embodiment, the proliferative disease is rheumatoid arthritis, psoriasis, or benign prostatic hyperplasia.

In still another embodiment, the therapeutically effective agent is an antiproliferative agent. More particularly, in certain embodiments, it is an alkylating drug, an antimetabolite, a microtubule inhibitor, a podophyllotoxin, an antibiotic, a nitrosourea, a hormone therapy, a kinase inhibitor, or an antiangiogenic agent. In further embodiments, it is carboplatin, doxorubicin, gemcitabine HCl, temolozolamide, cyclophosphamide, methotrexate, paclitaxel, etoposide, carmustine, cisplatin, tamoxifen, or interferon. In some embodiments, the kinase inhibitor is Iressa™ (ZD1839), Gleevec™ (STI-571), SU5416, or Tarceva™ (OSI-774).

Additional objects, features and advantages will become apparent to those skilled in the art from the following description and claims.

### BRIEF DESCRIPTION OF THE DRAWINGS

FIG. 1 is a graph that illustrates the effect of lometrexol on the IC₅₀ of tyrphostin AG1478 in A549 cells.

FIG. 2 is a graph which illustrates the effect of lometrexol on the IC₅₀ of indirubin-3′-monoxime in A549 cells.

FIG. 3 is a graph which illustrates the effect of different doses of lometrexol on the IC₅₀ of indirubin-3′-monoxime in A549 cells.

FIG. 4 is a graph that illustrates the effect of lometrexol on EGFR phosphorylation.

FIG. 5 is a graph that illustrates the effect of lometrexol on cellular ATP concentrations.

### DESCRIPTION OF THE SPECIFIC EMBODIMENTS

Definitions

The term “cancer” in an animal refers to the presence of cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone within an animal, or may circulate in the blood stream as independent cells, such as leukemic cells.

The phrase “a method of treating” or its equivalent, when applied to, for example, cancer refers to a procedure or course of action that is designed to reduce or eliminate the number of cancer cells in an animal, or to alleviate the symptoms of a cancer. “A method of treating” cancer or another proliferative disorder does not necessarily mean that the cancer cells or other disorder will, in fact, be eliminated, that the number of cells or disorder will, in fact, be reduced, or that the symptoms of a cancer or other disorder will, in fact, be alleviated. Often, a method of treating cancer will be performed even with a low likelihood of success, but which, given the medical history and estimated survival expectancy of an animal, is nevertheless deemed an overall beneficial course of action.

The “subject” is defined herein to include animals such as mammals, including, but not limited to, primates (e.g., humans), cows, sheep, goats, horses, dogs, cats, rabbits, rats, mice and the like.

The term “therapeutically effective agent” means a composition that will elicit the biological or medical response of a tissue, system, animal or human that is being sought by the researcher, veterinarian, medical doctor or other clinician.

The term “therapeutically effective amount” or “effective amount” means the amount of the subject compound that will elicit the biological or medical response of a tissue, system, animal or human that is being sought by the researcher, veterinarian, medical doctor or other clinician.

The term “pharmaceutically acceptable salts” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, oxalic, maleic, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, et. al., “Pharmaceutical Salts”, J. Pharmaceutical Science 66:1-19 (1977)). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are all intended to be encompassed within the scope of the present invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I or carbon-14 (¹⁴C). All isotopic variations of the compounds of the present invention, whether radioactive or not, are intended to be encompassed within the scope of the present invention.

“A combination amount sufficient”, “an effective combination amount”, “therapeutically effective combination amount”, or “an effective amount of the combination of” all refer to a combined amount of both lometrexol and the therapeutically effective agent that is effective to ameliorate symptoms associated with a particular disease. As used herein, the term “combination” of compound with the therapeutically effective agent means the two compounds can be delivered in a simultaneous manner, in combination therapy wherein lometrexol is administered first, followed by the therapeutically effective agent, as well as wherein the therapeutically effective agent is delivered first, followed by lometrexol. The desired result can be either a subjective relief of a symptom(s) or an objectively identifiable improvement in the recipient of the dosage.

The term “synergistic effective amount” refers to a combined amount of both lometrexol and an antiproliferative agent that is effective to cause a synergistic effect. Synergy is a biological phenomenon in which the effectiveness of two active components in a mixture is more than additive, i.e., the effectiveness is greater than the equivalent concentration of either component alone.

Description of Embodiments

Compositions

In one aspect, the present invention provides compositions and methods comprising the antineoplastic agent lometrexol and an antiproliferative agent. Advantageously, the compositions of the present invention provide significant clinical advantage over the use of a single agent alone. In certain proliferative disorders and patient populations, the described combinations of chemotherapeutic agents have increased efficacy over administration of either agent alone. Moreover, in some proliferative disorders and patient populations, the combination allows for the reduction in dosage of one or more of the agents used in combination therapy and, concomitantly, results in the reduction of adverse effects associated with each agent.

Lometrexol is the generic name given to the purine biosynthesis inhibitor 5,10-dideazatetrahydrofolic acid (DDATHF). This compound, along with its ability to inhibit glycinamide ribonucleotide transformylase (GARFT) and tumor growth, has been described by Taylor et al. in U.S. Pat. No. 4,684,653 and J. Med. Chem. 28:914-21 (1985). Additional processes for synthesis of lometrexol and isomeric variants are described in U.S. Pat. No. 4,902,796 and 4,927,828. A method for reducing toxicity of lometrexol by pre-treatment with folic acid is described in U.S. Pat. No. 5,217,974. Lometrexol has shown responses in early clinical trials for treatment of breast, bladder, and head and neck cancers, both with and without folic acid supplementation.

A wide range of antiproliferative agents can be used in the compositions and methods of the present invention. Antiproliferative agents are frequently categorized based on their mechanism of action (e.g., the nature of their activity on cell life cycle) and/or their chemical structure or properties. In preferred embodiments, the methods and compositions of the present invention comprise lometrexol, or a pharmaceutically acceptable salt thereof, combined with one or more antiproliferative agents from one or more of the categories set forth below. It is to be understood that the present invention contemplates combination therapy involving methods and compositions comprising lometrexol, or a pharmaceutically acceptable salt thereof, and therapeutic agents, e.g., antiproliferative agents, in addition to those discussed infra.

One category of suitable antiproliferative agents useful in the present invention is the alkylating agents, a group of highly reactive chemotherapeutics that form covalent linkages with nucleophilic centers (e.g., hydroxyl and carboxyl). Chemically, the alkylating agents can be divided into five groups: nitrogen mustards, ethylenimines, alkylsulfonates, triazenes, and nitrosureas. The nitrogen mustards are frequently useful in, for example, the treatment of chronic lymphocytic leukemia, Hodgkin's disease, malignant lymphoma, small cell lung cancer and breast and testicular cancer. Exemplary nitrogen mustards include chlorambucil, cyclophosphamide, ifosfamide, mechlorethamine, melphalan and uracil mustard. The ethylenimines, the most common of which is thiotepa, may be useful in bladder tumors and in breast and ovarian adenocarcinomas. The alkyl sulfonates are useful in the treatment of chronic myelogenous leukemia and other myeloproliferative disorders. Exemplary alkyl sulfonates include busulfan and piposulfan. The triazines, which include, e.g., dacarbazine, are useful in the treatment of malignant melanomas and sarcomas. Temozolomide, an analog of dacarbazine, may also be used in the methods and compositions of the present invention. Finally, the nitrosureas are especially useful against brain tumors, but also are effective for, e.g., multiple myeloma, malignant melanoma, and lymphoma. Exemplary nitrosureas include carmustine and lomustine.

Another category of antiproliferative agents suitable for use in the present invention is the antimetabolites, structural analogs of normally occurring metabolites that interfere with normal nucleic acid biosynthesis. This category of agents may be subdivided into the folic acid analogs, purine analogs and pyrimidine analogs based on the function of the metabolite with which the agent interferes. The most common folic acid analog is methotrexate, useful in the treatment of choriocarcinoma, leukemias, neoplasms and psoriasis. The purine analogs, such as mercaptopurine, thioguanine and azathioprine, may be useful in leukemias. The pyrimidine analogs are useful in the treatment of, for example, leukemia and carcinomas of the gastrointestinal tract, mammary gland, and bladder. Exemplary pyrimidine analogs include fluorouracil (5-FU), UFT (uracil and ftorafur), capecitabine, gemcitabine and cytarabine.

The vinca alkaloids, natural product-based agents that exert their cytotoxicity by binding to tubulin, represent another category of antiproliferative agents suitable for use in the present invention. The vinca alkaloids are useful in, for example, the treatment of lymphomas, leukemias, and lung, breast, testicular, bladder and head and neck cancers. Exemplary agents include vinblastine, vincristine, vinorelbine and vindesine. The taxanes, agents which promote microtubule assembly, and the podophyllotoxins, agents which inhibit topoisomerases, represent related categories of antiproliferative agents that may be useful in the methods and compositions of the present invention. Exemplary taxanes include paclitaxol and docetaxol, which are useful in breast and lung cancers, among others. Exemplary podophyllotoxins include etoposide (useful in, for example, lymphoma and Hodgkin's disease), teniposide, ironotecan (useful in, for example, colon, rectal and lung cancer) and topotecan, the latter two which act via inhibition of topoisomerase I.

Antineoplastic antibiotics represent another category of antiproliferative agents useful in the methods and compositions of the present invention. These agents exert their effects by binding to or complexing with DNA. Exemplary agents include daunorubicin, doxorubicin, epirubicin, mitoxantrone, mitomycin, dactinomycin, plicamycin, and bleomycin. The antibiotics are useful in a diverse range of disorders, including Hodgkin's disease, leukemia, lymphoma, and lung cancer.

The methods and compositions of the present invention may comprise other antiproliferative agents, including the platinum complexes (e.g., cisplatin and carboplatin, which are especially useful in the treatment of lung, head and neck, ovarian and breast cancer); enzymes (e.g., L-asparaginase); hormone-related therapy hormone (e.g., tamoxifen, leuprolide, flutamide, megesterol acetate, diethylstilbestrol, prednisone and estradiol cypionate); hydroxyurea; methylhydrazine derivatives such as procarbazine; adrenocortical suppressants, e.g., mitotane, aminoglutethimide; aromatase inhibitors (e.g., anastrozole); and biologic response modifiers (e.g., interferon-A).

Furthermore, the methods and compositions of the present invention may comprise antiproliferative agents that result from the combination of two or more agents including, for example, prednimustine (a conjugate of prednisone and chlorambucil) and estramustine (a conjugate of nornitrogen mustard and estradiol).

In preferred embodiments, the compositions and methods of the present invention comprise lometrexol in combination with carboplatin, doxorubicin, gemcitabine, paclitaxel, or temozolomide.

The methods and compositions of the present invention may comprise lometrexol in combination with a kinase inhibitor. Although the present invention is not limited to any particular kinase, kinase inhibitors contemplated for use include Iressa™ (ZD1839; Astra Zeneca); Gleevec™ (STI-571 or imatinib mesylate; Novartis); SU5416 (Pharmacia Corp./SUGEN); and Tarceva™ (OSI-774; Roche/Genentech/OSI Pharmaceuticals). Multiple kinases have been implicated in neoplasia and investigated as potential therapeutic targets. Kinases can generally be classified into two major types, those which phosphorylate substrates on serine/threonine residues and those which phosphorylate substrates on tyrosine residues. Ser/thr kinases include the receptor ser/thr kinase TGF-β receptor and nonreceptor ser/thr kinases, such as the MAP kinases, PKC, PKA, and the cyclin-dependent kinases (CDKs) that regulate the cell cycle. Since dysregulated CDK activity is a hallmark of neoplasia, numerous recent studies have investigated inhibitors and modulators of these proteins as novel therapeutic agents for cancer (see, Sausville, et al., Pharmacol Ther., 82:285-92 (1999)).

In certain embodiments, the inhibitors used in the combination therapy of this invention target kinases involved in cell cycle regulation. Most preferably, the kinase inhibitors are tyrphostin AG490 (2-cyano-3-(3,4-dihydroxyphenyl)-N-(benzyl)-2-propenamide), which inhibits the activation of CDK2 (Kleinberger-Doron, et al. Exp. Cell. Res. 241:340-51 (1998)); alsterpaullone, which shows high CDK1/cyclin B inhibitory activity and high in vitro antitumor activity (Schultz, et al, J. Med. Chem. 42:2909-19 (1999)); and indirubin-3′-monoxime, which directly inhibits CDK2 kinase activity (Hoessel, et. al., Nat. Cell. Biol. 1:60-67 (1999)).

Certain tyrosine kinases are also implicated in neoplasia. Tyrosine kinases include those with transmembrane regions and extracellular portions, known as receptor tyrosine kinases (RTKs), and nonreceptor tyrosine kinases, which lack an extracellular domain. Four different structural classes of receptor tyrosine kinases are evident. Type I is exemplified by the epidermal growth factor receptor (EGFR), type II by the insulin receptor, type III by the platelet-derived growth factor receptor, and type IV by the fibroblast growth factor receptor. Nonreceptor tyrosine kinases are exemplified by the src and janus families. RTKs, nonreceptor tyrosine kinases, and other proteins in the RTK signalling pathway play central roles in cell growth and differentiation and account for a high proportion of known oncogenes.

In certain embodiments, the inhibitors used in the combination therapy of this invention target RTKs involved in growth factor signalling pathways. Most preferably, the kinase inhibitor is genistein, a broad spectrum growth factor kinase inhibitor; tyrphostin AG1478 (4-(3-chloroanilino)-6,7-dimethoxyquinazoline), an EGFR-specific kinase inhibitor; or tyrphostin AG490 (2-cyano-3-(3,4-dihydroxyphenyl)-N-(benzyl)-2-propenamide), a compound which targets JAK2, a kinase that transmits IL6 cellular differentiation and growth signals to the nucleus. In other preferred embodiments, the kinase inhibitor is Iressa™ (ZD1839), Gleevec™ (STI-571), SU5416, or Tarceva™ (OSI-774).

Without being bound by any particular theory, lometrexol's ability to potentiate a wide range of other antiproliferative agents is thought to arise from its ability to lower ATP concentrations and relative levels compared to other nucleotides (see, Sokoloski et al., Oncol Res 5:293(1993)). In particular, the significant lowering of ATP without a concomitant decrease in other nucleotides generates a nucleotide pool imbalance. Numerous investigators have shown that incorporation of unnatural nucleotides increases when there is a nucleotide pool imbalance. Accordingly, the effect of nucleotide mimics like gemcitabine can be potentiated by lometrexol. Furthermore, the imbalance in the nucleotide pools generated by lometrexol causes increased mismatch incorporations when the cell's DNA-repair machinery is activated by other chemotherapeutic agents that induce apoptosis (e.g., alkylating agents, free radical generating/topoisomerase inhibitors, agents that prevent DNA methylation). The resulting increased need for repair thus leads to even more rapid induction of apoptosis. Since tubulin dynamics are strongly affected by GTP and ATP hydrolysis, the alteration in the purine nucleotide pools also enhances the efficacy of chemotherapeutics that interfere with normal tubulin dynamics. Finally, the lowering of ATP levels by lometrexol allows kinase inhibitors acting as antiproliferative agents to more effectively compete for their target kinases.

Thus this invention provides combinations of lometrexol and other antiproliferative agents (e.g., carboplatin, doxorubicin, gemcitabine HCl, paclitaxel, temolozolamide) that can provide a clinical advantage. Additionally, combinations of lometrexol and multiple kinase inhibitors have now been evaluated in cellular proliferation studies and shown to potentiate proliferative effects of the kinase inhibitors.

Analysis of Compositions

In vitro assays can be used to establish that the subject compositions inhibit proliferation. This inhibition is preferably 20%, 30%, 40%, 50%, or most preferably 50% or higher. The term “proliferative” refers to any effect which changes the rate of cell growth.

Preferred compositions of this invention may be evaluated in vitro for their ability to inhibit proliferation by any method known to those of skill in the art, preferably as described in Ahmed et al. (J. Immunol. Methods 170:211 (1994)). In preferred embodiments, the potentiation of a proliferative effect is assayed by measuring enzyme levels (i.e., the MTT assay (3-(4,5-dimethylthiazol-2-yl)-5-(3-carboxymethoxyphenyl)-2-(4-sulfophenyl)2H-tetrazolium salt) which forms a colored formazan product in the presence of active mitochondrial dehydrogenases within the cell) or cell counting.

Established animal models to evaluate proliferative effects of compositions are also known in the art. For example, compounds can be evaluated for their ability to inhibit the growth of human tumors grafted into immunodeficient mice using methodology similar to that described by Rygaard and Povlsen (Acta Pathol. Microbiol. Scand. 78:758 (1969)) and Giovanella and Fogh (Adv. Cancer Res. 44:69 (1985)).

Formulations

The compositions provided above can be formulated in a variety of formats well-known to those of skill in the art (see, Remington's Pharmaceutical Sciences, A.R. Genaro (ed.), 19th ed., Mark Publishing Co., Easton, Pa. (1995)).

The compositions of the invention and the pharmaceutically acceptable salts thereof can be administered in any effective way such as via oral, parenteral or topical routes.

Generally, the compounds are administered in dosages ranging from about 2 mg up to about 2,000 mg per day, although variations will necessarily occur depending on the disease target, the patient, and the route of administration. Preferred dosages are administered intravenously or orally in the range of about 30 to 100 mg/m² of body surface area (BSA) for lometrexol sodium, a range of milligrams that is a function of an area under the plasma concentration versus time curve of 4 to 7 mg/mL-min and the particular subject's glomerular filtration rate for carboplatin, 40 to 75 mg/m² BSA for doxorubicin, 800 to 1250 mg/m² BSA for gemcitabine HC1, 175 to 225 mg/m² BSA for paclitaxel, and 100 to 200 mg/m² BSA for temozolamide.

Effective combination amounts for various uses will depend on, for example, the particular antiproliferative agent, the manner of administration, the weight and general state of health of the patient, and the judgment of the prescribing physician. In preferred embodiments, the composition or formulation to be administered will contain a quantity of lometrexol or antiproliferative agent less than the amount that would treat the proliferative disorder if administered alone. Combination therapy can allow for the reduction in dosage of all agents used in the therapy and reduce the side effects associated with each agent.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

In one embodiment, the invention provides the subject compositions combined with a pharmaceutically acceptable excipient such as sterile saline or other medium, water, gelatin, an oil, etc. to form pharmaceutically acceptable compositions. The compositions and/or compounds may be administered alone or in combination with any convenient carrier, diluent, etc. and such administration may be provided in single or multiple dosages. Useful carriers include solid, semi-solid or liquid media including water and non-toxic organic solvents.

In another embodiment, the invention provides the subject compounds in the form of a pro-drug, which can be metabolically or chemically converted to the subject compound by the recipient host. A wide variety of pro-drug formulations are known in the art.

The compositions may be provided in any convenient form including tablets, capsules, lozenges, troches, hard candies, powders, sprays, creams, suppositories, etc. As such, the compositions, in pharmaceutically acceptable dosage units or in bulk, may be incorporated into a wide variety of containers. For example, dosage units may be included in a variety of containers including capsules, pills, etc.

Since the present invention has an aspect that relates to a combination of active ingredients which can be administered separately, the invention also relates to combining separate pharmaceutical compositions in kit form. In a preferred embodiment, the kit comprises two separate pharmaceutical compositions: lometrexol and a second compound such as an antiproliferative agent as described above. The kit comprises a container for containing the separate components such as a divided bottle or a divided foil packet, however, the separate components can also be contained within a single, undivided container. Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

Methods of Treating Proliferative Disorders

The invention provides methods of using the subject compositions to treat disease or provide medicinal prophylaxis, to treat proliferative disorders, etc. These methods generally involve contacting the cell with or administering to the host an effective amount of the subject compounds or pharmaceutically acceptable compositions. In one embodiment, treatment is carried out using a composition comprising lometrexol and at least one other antiproliferative agent. More particularly, this invention provides a method for the treatment of proliferative disorders, comprising administering to a subject in need of such treatment an effective amount of a composition comprising lometrexol or a pharmaceutically acceptable salt thereof and one or more therapeutically effective agents or pharmaceutically acceptable salts thereof. In another embodiment, treatment comprises separate administration of the two agents. More particularly, this invention provides a method for the treatment of proliferative disorders, comprising administering to a subject in need of such treatment an effective first amount of lometrexol or a pharmaceutically acceptable salt thereof and an effective second amount of one or more therapeutically effective agents or pharmaceutically acceptable salts thereof.

i. Combination Composition

In this embodiment of the invention, a composition of lometrexol and an antiproliferative agent is administered to a patient in need of treatment. The amount of each agent will typically be less than an amount that would produce a therapeutic effect if administered alone. The precise method of administration will depend on the patient, particular antiproliferative agent, and the judgment of the clinician, but will preferably be intravenous or oral.

ii. Compositions Used Separately (Administered Either Simultaneously or Sequentially)

In this embodiment of the invention, lometrexol and the antiproliferative agent are administered separately. Those of skill in the art will readily understand that the two compositions can be administered simultaneously. Alternatively, lometrexol is administered first, followed by the antiproliferative agent within a month, more preferably within a week, and most preferably within a day. In yet another aspect, the antiproliferative agent is delivered first, followed by lometrexol within a month, more preferably within a week, or most preferably within a day.

Administration of these compositions can be via any method which provides systemic exposure to the compounds of this invention. These methods include oral routes, parenteral, intraduodenal routes, etc. Generally, the compounds of the present invention are administered in single (e.g., once daily) or multiple doses. The compounds of the present invention are generally administered in the form of a pharmaceutical composition comprising a pharmaceutically acceptable carrier or diluent. Thus, the compounds of this invention can be administered individually or together in any conventional oral, parenteral or transdermal dosage form. Of course, other forms of administration of the active ingredients, as they become available, are contemplated, such as by nasal spray, transdermally, by suppository, by sustained release dosage form, by IV injection, etc. Any form of administration will work so long as the proper dosages are delivered without destroying the active ingredient.

Treatment cycles may be continued until a clinical response is achieved or until intolerable side effects are encountered. The dosages of lometrexol and/or the antiproliferative agent may be increased with each new treatment cycle, provided intolerable side effects are not encountered. The dosages may also be decreased, if intolerable side effects are encountered.

The actual preferred course of therapy can vary according to, inter alia, the mode of administration of lometrexol, the particular formulation of the antiproliferative agent being utilized, the mode of administration of the agents, the particular disease being treated and the particular host being treated. The optimal course of therapy for a given set of conditions can be ascertained by those skilled in the art using a conventional course of therapy determination tests and in view of the information set out herein.

The effectiveness of treatment may be determined by controlled clinical trials, generally in Phase II and Phase III clinical trials. Patients having cancer with measurable or evaluable tumors will be included in the study. A measurable tumor is one that can be measured in at least two dimensions such as a lung tumor surrounded by aerated lung, a skin nodule, or a superficial lymph node. An evaluable tumor is one that can be measured in one dimension such as a lung tumor not completely surrounded by aerated lung or a palpable abdominal or soft tissue mass that can be measured in one dimension. Tumor markers which have been shown to be highly correlated with extent of disease will also be considered to provide an evaluable disease, such as PSA for prostate cancer, CA-125 for ovarian cancer, CA-15-3 for breast cancer, etc.

The tumor will be measured or evaluated before and after treatment by whatever means provides the most accurate measurement, such as CT scan, MRI scan, Ultrasonography, etc. New tumors or the lack thereof in previously irradiated fields can also be used to assess the anti-tumor response. The criteria for evaluating response will be similar to that of the WHO Handbook of Reporting Results of Cancer Treatment, WHO Offset Publication 1979, 49-World Health Organization, Geneva. The following results are defined for uni- and bi-dimensionally measurable tumors.

Complete response: Complete disappearance of all clinically detectable malignant disease determined by two observations not less than four weeks apart.

Partial response: For bidimensionally measurable tumors, a decrease of at least 50% in the sum of the products of the largest perpendicular diameters of all measurable tumors as determined by two observations not less than four weeks apart. For unidimensionally measurable tumors, a decrease by at least 50% in the sum of the largest diameters of all tumors as determined by two observations not less than four weeks apart. In cases where the patient has multiple tumors, it is not necessary for all tumors to have regressed to achieve a partial response as defined herein, but no tumor should have progressed and no new tumor should appear.

Stable disease: For bidimensionally measurable tumors, less than a 50% decrease to less than a 25% increase in the sum of the products of the largest perpendicular diameters of all measurable tumors. For unidimensionally measurable tumors, less than a 50% decrease to less than a 25 % increase in the sum of the diameters of all tumors. No new tumors should appear.

No clinical response (i.e., progressive disease) is defined as an increase of more than 50% in the product of the largest perpendicular diameters for at least one bidimensionally measurable tumor, or an increase of more than 25% in measurable dimension of at least one unidimensionally measurable tumor.

Of course elimination or alleviation of other known signs or symptoms of cancer, especially those listed previously can also be used to evaluate the effectiveness of this invention.

Another aspect of this invention is the treatment of cancer with reduced side effects normally associated with lometrexol. This objective can be achieved by administration of lower doses of the two active ingredients or by shorter duration of dosing.

The most common side effects of lometrexol are anorexia, weight loss, mucositis, leukopenia, anemia, hypoactivity, and dehydration.

The following examples are offered by way of illustration and not by way of limitation.

### EXAMPLES

### Example 1

### Lometrexol Potentiates the Antiproliferative Effect of Kinase Inhibitors

This example demonstrates that kinase inhibitor activity is enhanced by the co-administration of lometrexol, and that a likely mechanism for this effect is reduction of available ATP.

Lometrexol Potentiates the Activity of the Kinase Inhibitor AG1478

A549 and MDA-MB-231 cells were obtained from the ATCC. Cells were propagated in 75 cm² flasks in RPMI 1640 medium with 10% fetal bovine serum, 1% penicillin/streptomycin, and 1% glutamine. To initiate the experiment, cells were plated into 96-well plates at 3000 cells/well with the top and bottom rows containing media only as a negative control. The cells were incubated at 37° C. for 15 hours to allow the cells to adhere. After 15 hours, freshly prepared solutions of lometrexol and the appropriate kinase inhibitor (AG1478 or indirubin-3′-monoxime) at the desired dose were added to the media in varying concentrations in a 5×8 matrix of concentrations. Each plate also contained wells that contained medium only and no added compound. The cells were then incubated for 48 hours with the compounds. The relative cell number was determined using the cell proliferation assay reagent MTT. This reagent is converted by mitochondrial dehydrogenases to a colored formazan product that is then quantified by spectrophotometric analysis. All the wells were corrected for the background using the absorbance determined from the wells that did not contain cells. The relative effect of the compounds was determined by the percentage of the untreated control cells.

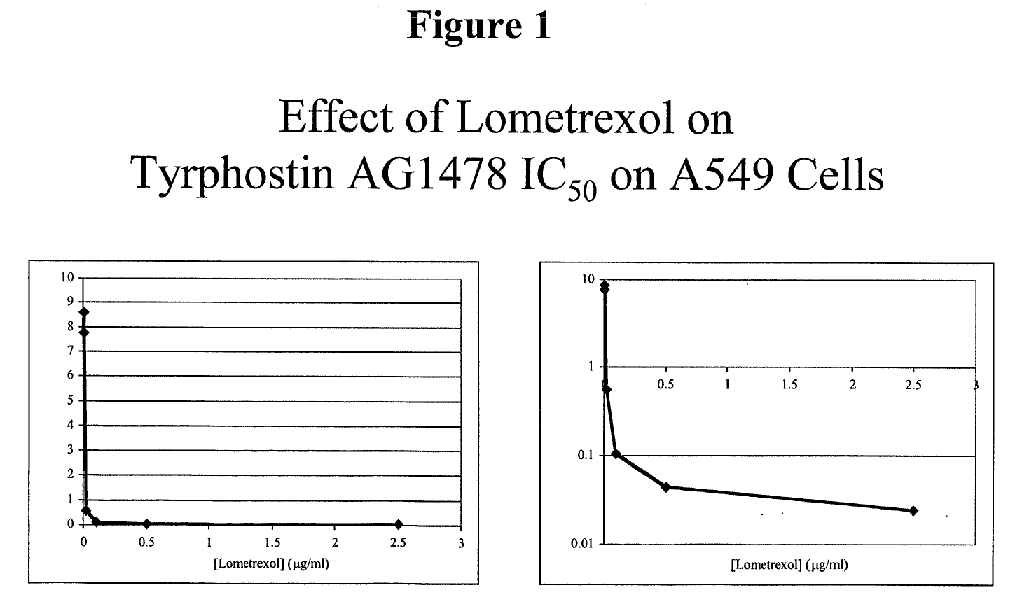

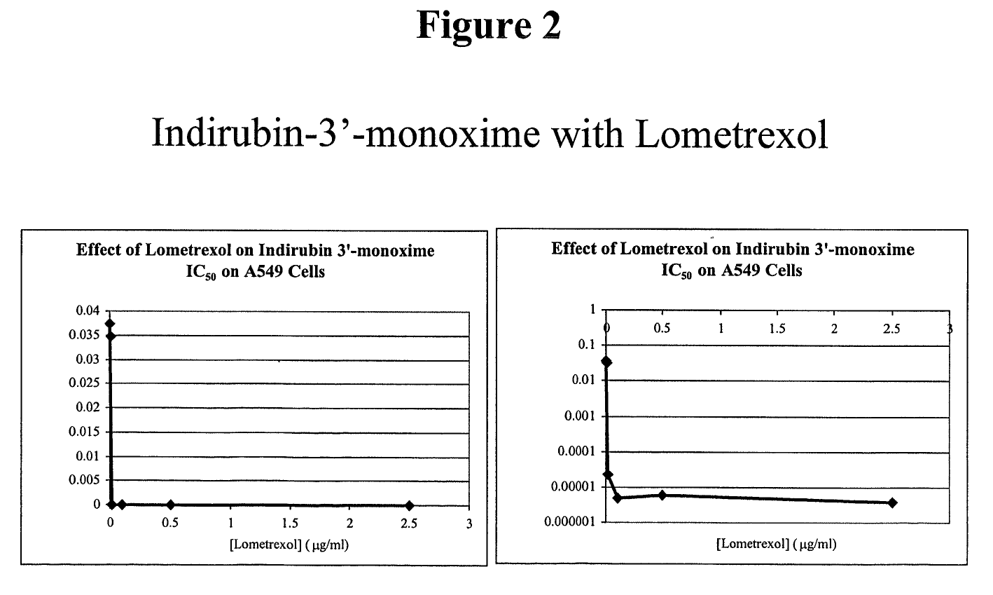

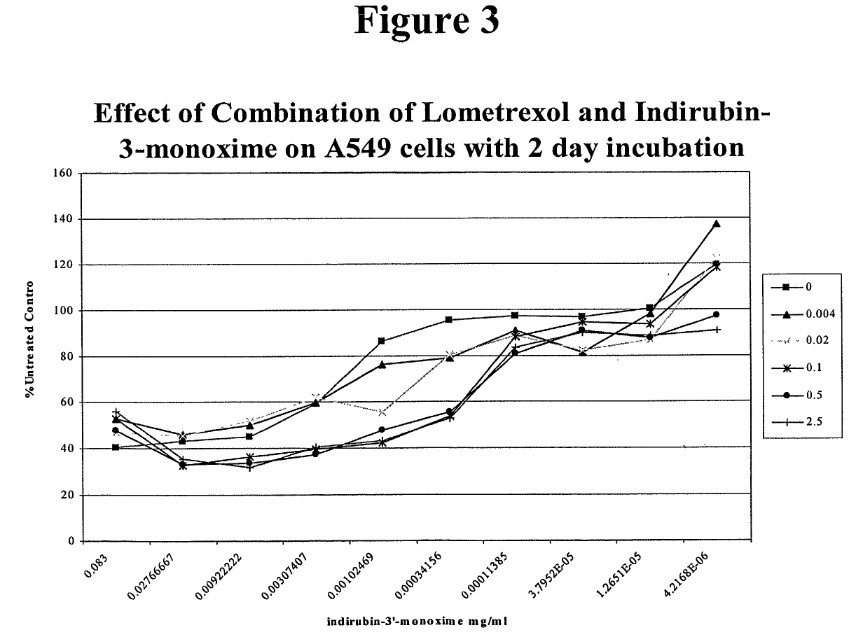

FIG. 1 shows the IC₅₀ of tyrphostin AG1478 in A549 cells in the presence of increasing concentrations of lometrexol. The IC₅₀ value was calculated from the untreated cells. The observed decrease in IC₅₀ is consistent with a potentiation of the growth inhibitory effect of tyrphostin AG1478. FIG. 2 shows the IC₅₀ of indirubin-3′-monoxime in A549 cells in the presence of increasing concentrations of lometrexol. The IC₅₀ value was calculated from the untreated cells. The observed decrease in IC₅₀ is consistent with a potentiation of the growth inhibitory effect of indirubin-3′-monoxime. FIG. 3 shows the growth inhibition of A549 cells by indirubin-3′-monoxime with varying concentrations of lometrexol. The data are presented as % growth relative to cells treated with lometrexol alone. The total incubation time with the compounds was 48 hours.

Lometrexol Decreases the Ability Of EGF to be Phosphorylated

The A549 lung adenocarcinoma cell line was grown under normal cell culture conditions. The cells were plated in 100 mm² dishes in RPMI 1640 culture medium containing 1% penicillin/streptomycin, 1% glutamine and 10% fetal bovine serum. The cells were allowed to grow to >95% confluence. Thereafter, the cells were rinsed twice with phosphate-buffered saline followed by the addition of low-serum medium (RPMI 1640 with 1% penicillin/streptomycin and 0.5% fetal bovine serum).

Lometrexol was added in amounts sufficient to achieve incubation concentrations of 0.004, 0.02, 0.1, 0.5 and 2.5 μg/ml. The cells were incubated in the low-serum medium with lometrexol for 24 hours. After the 24-hour incubation, the concentration of fetal bovine serum was increased to 10%, and 50 ng/ml EGF was added to the cells in the presence of lometrexol. The cells were incubated with these stimulants (fetal bovine serum and EGF) for 3 hours. After the EGFR stimulation, the cells were rinsed with cold phosphate-buffered saline, scrapped off the dishes, and lysed by douncing. The total EGFR was isolated from the bulk cell lysates by immunoprecipitation using an antibody to the cytoplasmic domain (Chemicon, Temecula, Calif.). The cell lysates were standardized by using 400 g of total protein in each reaction. The immunoprecipitated protein was separated by SDS-PAGE, and the amount of phosphorylated EGFR was determined by western blot analysis of the resulting gel using chemiluminescent detection. The blot was probed with an antibody that is specific for the activated, phosphorylated form of EGFR (Chemicon, Temecula, Calif.), and the resulting photographic film was analyzed by densitometry.

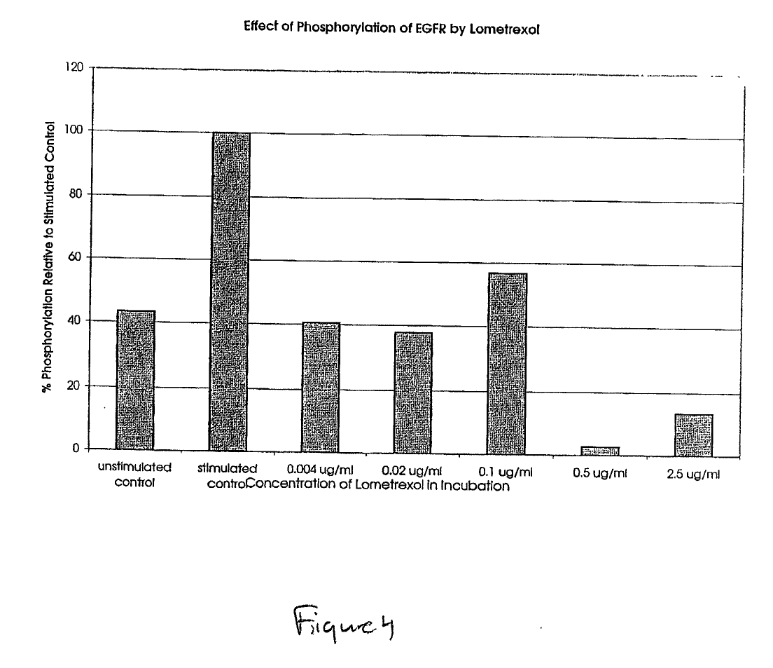

As depicted in FIG. 4, there was approximately a 2.3-fold increase in the amount of phosphorylated EGFR with stimulation compared to the cells that were not stimulated with fetal bovine serum and EGF for 3 hours. Lometrexol decreased the amount of phosphorylated EGFR, even in the presence of stimulation. Lometrexol treatment at 0.004, 0.02 and 0.1 μg/ml decreased the relative amount of phosphorylated-EGFR per mg protein in the cell lysate to between 37-56% of control. The decrease in phosphorylated EGFR was even greater at concentrations of 0.5 and 2.5 μg/ml lometrexol, which lowered the relative phosphorylated-EGFR to as little as 3% of the activated-control. These data are consistent with the growth-inhibition potentiation observed on the A549 cells by these concentrations of lometrexol on tyrphostin 1478, and thus support the hypothesis that lometrexol decreases ATP available for phosphorylation reactions in cells.

Lometrexol Reduces Adenosine Triphosphate Stores

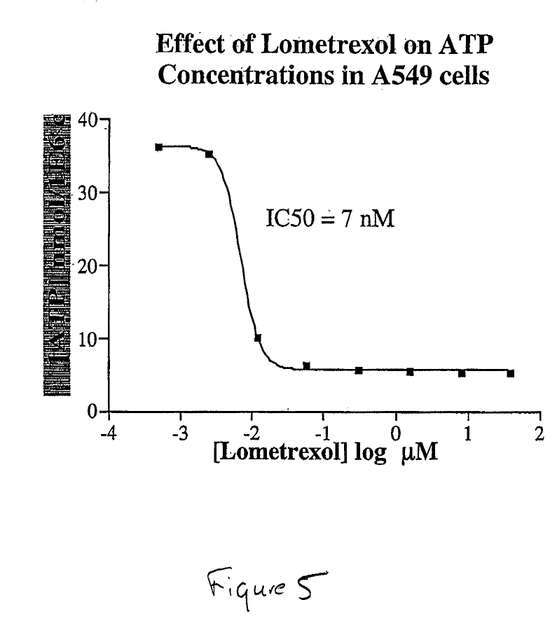

A549 cells were propagated in 75 cm² flasks in RPMI 1640 medium with 10% fetal bovine serum, 1% penicillin/streptomycin, and 1% glutamine. To initiate the experiment, cells were plated into 96-well plates at 3000 and 10,000 cells/well with the top and bottom rows containing media only as a negative control. The cells were incubated at 37° C. for 15 hours to allow the cells to adhere. After 15 hours, freshly prepared solutions of lometrexol at the desired dose were added to the media in varying concentrations. Each plate also contained wells that contained medium only and no added compound. The cells were then incubated for 24 hours and the amount of ATP was determined by a luminescent method, CellTiter Glo™ (Promega, Madison, Wis.). Concurrently, the cell number was determined in duplicate rows so that the relative ATP concentration per number of cells could be determined. As depicted in FIG. 5 (average of triplicate measurements), the ATP concentration per million cells was decreased in cells treated with lometrexol at concentrations similar to those where the EGFR phosphorylation was effected and at concentrations similar to those which potentiate kinase inhibitors.

Although a precise understanding of the mechanism by which lometrexol potentiates kinase inhibitor activity is not necessary in order to practice the present invention, as alluded to above such potentiation is believed to result, at least in part, from lometrexol's effect on reducing ATP levels. In an environment where ATP levels have been reduced, those kinase inhibitors that are ATP competitive have less ATP against which to compete, and thus lometrexol would potentiate their activity. This mechanism is supported by the fact that in a cell line known to be sensitive to EGF stimulation (A549 cells as used above; see Robinson et al., J. Steroid Biochem. Mol. Biol. 37:883 (1990)), lometrexol potentiation is observed. However, there is no observed potentiation in cells showing only mild growth inhibition by EGF (MDA-MB-231 cells; see Davidson et al., Mol. Endocrinol. 1:216 (1987)).

The following examples illustrate methods to establish the efficacy, maximum tolerated dose, recommended dose, toxicity, and pharmacokinetics of certain therapeutically effective agents in combination therapy with lometrexol.

### Example 2

### A Phase I Trial of Lometrexol Sodium and Carboplatin Administered Intravenously Every 21 Days in Conjunction with Oral Folic Acid in Patients with Solid Tumors

This is a Phase I, open-label study of the combination of lometrexol sodium and carboplatin, a platinum-containing compound, administered intravenously (IV) to patients with locally advanced/metastatic cancer. Patients who are eligible for this study have a diagnosis of locally advanced/metastatic solid tumor that has failed conventional treatment or for which no standard therapy is available. A sufficient number of patients are enrolled at up to six dose levels to determine the maximum tolerated dose (MTD) of lometrexol sodium and carboplatin given in conjunction with folic acid. Approximately 12 to 42 patients are entering the study.

The study objectives are as follows:

(1) To determine the MTD of lometrexol sodium and carboplatin administered every 21 days in conjunction with folic acid in patients with solid tumors.

(2) To establish a recommended dose of lometrexol sodium and carboplatin given with folic acid for study in Phase II trials.

(3) To determine the quantitative and qualitative toxicities of lometrexol sodium and carboplatin when given in conjunction with folic acid on this schedule.

(4) To determine the plasma concentrations of lometrexol sodium and carboplatin that are achieved on this schedule, and to relate their pharmacokinetics to toxicity outcome.

(5) To document the antitumor activity of lometrexol sodium and carboplatin when given in conjunction with folic acid on this schedule.

During each 21-day cycle, patients take folic acid 5 mg orally once daily starting 7 days prior to the start of each cycle and continuing for 7 days after receiving lometrexol sodium and carboplatin. On Day 1 of each cycle, patients receive an intravenous bolus dose of lometrexol sodium over approximately 30 to 60 seconds followed by IV infusion of carboplatin over approximately 15 to 30 minutes. Treatment is given on an outpatient basis every 21 days. Patients are allowed to receive multiple cycles as long as eligibility and re-treatment criteria continue to be met, toxicity is acceptable, and there is no evidence of disease progression. After the initial dose, adjustments of the dose of lometrexol sodium and carboplatin are allowed based on individual patient tolerance. Blood and urine sampling are performed during Cycle 1 for lometrexol sodium and carboplatin pharmacokinetics.

Dosages and Administration

Lometrexol sodium for injection is supplied for the study as a lyophilised off-white crystalline solid in single-use vials containing either 50 mg or 200 mg of lometrexol sodium. Vials are stored at room temperature and protected from light. Lometrexol sodium is reconstituted with 0.9% sodium chloride for injection to a concentration of approximately 10 mg/mL. Vials do not contain a preservative; therefore, the drug should be used within 1 hour of reconstitution. Lometrexol sodium is not known to be a vesicant. Lometrexol sodium is administered on Day 1 of every 21-day cycle as a rapid (i.e., 30 to 60 seconds) intravenous bolus immediately prior to the dose of carboplatin. At each dose level shown in Table 1, the appropriate dose of lometrexol sodium is based on the patient's actual calculated body surface area (BSA) at the beginning of each cycle. Note: If the patient's BSA is >2.0 m² the dose is calculated using a maximum BSA of 2.0 m².

Carboplatin is commercially available from the manufacturer. The drug is reconstituted according to the instructions on the package and used as directed. Normal saline is not used to further dilute the drug for intravenous administration. Aluminum reacts with carboplatin causing precipitate formation and loss of potency. Therefore, needles or intravenous sets containing aluminum parts that may come in contact with the drug are not used for the preparation or administration of carboplatin.

Doses in this study are calculated according to the Calvert Formula, where AUC is area under the plasma concentration versus time curve (mg/mL·min) and GFR is glomerular filtration rate (estimated by creatinine clearance [mL/min]).

Total Dose (mg)=(target AUC)×(GFR+25)

At each dose level shown in Table 1, the appropriate dose of carboplatin is based on the patient's GFR using the serum creatinine measured at the beginning of each 21-day cycle. Note: Carboplatin is infused over 15 to 30 minutes immediately following the bolus dose of lometrexol sodium.

The dose levels and number of patients at each level are shown in Table 1.

TABLE 1
Dose Levels

| | Carboplatin AUC | Lometrexol | |
|---|---|---|---|
| Dose Level | (mg/mL · mm) | (mg/m²) | Number of Patients |
| −1 | 4 | 30 | 3 to 6 |
| 1 | 4 | 50 | 3 to 6 |
| 2 | 5 | 50 | 3 to 6 |
| 3 | 5 | 75 | 3 to 6 |
| 4 | 5 | 100 | 3 to 6 |
| 5 | 6 | 100 | 3 to 6 |
| 6 | 7 | 100 | 3 to 6 |

### Example 3

### A Phase I Trial of Lometrexol Sodium and Doxorubicin Administered Intravenously Every 21 Days in Conjunction with Oral Folic Acid in Patients with Solid Tumors

This is a Phase I, open-label study of the combination of lometrexol sodium and doxorubicin, an anthracycline antibiotic, administered intravenously to patients with locally advanced/metastatic cancer. This dose-finding study provides a recommendation for the dosing of this drug combination in future studies. Patients who are eligible for this study have a diagnosis of locally advanced/metastatic solid tumor that has failed conventional treatment or for which no standard therapy is available. A sufficient number of patients are enrolled at up to five dose levels to determine the MTD of lometrexol sodium and doxorubicin given in conjunction with folic acid. Approximately 12 to 42 patients are enrolling in the study.

The study objectives are as follows:

(1) To determine the MTD of lometrexol sodium and doxorubicin administered every 21 days in conjunction with folic acid in patients with solid tumors.

(2) To establish a recommended dose of lometrexol sodium and doxorubicin given in conjunction with folic acid for study in Phase II trials.

(3) To determine the quantitative and qualitative toxicities of lometrexol sodium and doxorubicin when given in conjunction with folic acid on this schedule.

(4) To determine the plasma concentrations of lometrexol sodium and doxorubicin that are achieved on this schedule, and to relate their pharmacokinetics to toxicity outcome.

(5) To document the antitumor activity of lometrexol sodium and doxorubicin when given in conjunction with folic acid on this schedule.

During each 21-day cycle, patients take folic acid 5 mg orally once daily starting 7 days prior to the start of each cycle and continuing for 7 days after receiving lometrexol sodium and doxorubicin. On Day 1 of each cycle, an intravenous bolus dose of lometrexol sodium is administered (over 30 to 60 seconds) followed immediately by a slow attended IV infusion of doxorubicin over 15 minutes. Treatment is given on an outpatient basis every 21 days. Patients receive multiple cycles as long as eligibility and re-treatment criteria continue to be met, toxicity is acceptable, and there is no evidence of disease progression. After the initial dose, adjustments of the dose of lometrexol sodium and doxorubicin are allowed based on individual patient tolerance. Blood and urine sampling is performed during Cycle 1 for lometrexol sodium and doxorubicin pharmacokinetics.

Dosages and Administration

Lometrexol sodium is prepared and administered as described above in Example 2.

Doxorubicin HCl is available commercially available from several manufacturers in liquid or lyophilised form. The lyophilised powder is stable for 2 years when stored at room temperature and away from direct light. The commercial solution formulations must be stored under refrigeration. The lyophilised drug is reconstituted with either sterile water for injection or 0.9% sodium chloride. The reconstituted solution should be used straight away, but if not used, may be stored for up to 24 hours. Doxorubicin HCl is physically incompatible with a number of drugs. Therefore, a 5 to 10 mL flush of D5W or normal saline is given before and after doxorubicin. For bolus dosing every 3 weeks, a dose range of 60 to 75 mg/m² is preferred. At each dose level shown in Table 2, the appropriate dose of doxorubicin is based on the patient's actual calculated body surface area (BSA) at the beginning of each 21-day cycle. Note: Doxorubicin is administered as a slow attended intravenous infusion over 15 minutes immediately following the dose of lometrexol sodium on Day 1 of each cycle.

The dose levels and number of patients at each level are shown in Table 2.

TABLE 2
Dose Levels

| | Lometrexol Sodium | | Number of |
|---|---|---|---|
| Dose Level | (mg/m²) | Doxorubicin (mg/m²) | Patients |
| −1 | 30 | 40 | 3 to 6 |
| 1 | 30 | 50 | 3 to 6 |
| 2 | 50 | 50 | 3 to 6 |
| 3 | 75 | 50 | 3 to 6 |
| 4 | 90 | 50 | 3 to 6 |
| 5 | 100 | 60 | 3 to 6 |

### Example 4

### A Phase I Trial of Lometrexol Sodium and Gemcitabine HC1 Administered Intravenously in Conjunction with Oral Folic Acid in Patients with Solid Tumors

This is a Phase I, open-label study of the combination of lometrexol sodium and gemcitabine HCl, a nucleoside analogue, administered intravenously to patients with locally advanced/metastatic cancer. Patients who are eligible for this study have a diagnosis of locally advanced/metastatic solid tumor that has failed conventional treatment or for which no standard therapy is available. A sufficient number of patients are enrolled at up to five dose levels to determine the maximum tolerated dose (MTD) of lometrexol sodium and gemcitabine HCl given in conjunction with folic acid. Approximately 12 to 42 patients are entering this study.

The study objectives are as follows:

(1) To determine the MTD of lometrexol sodium and gemcitabine HCl administered in conjunction with folic acid in patients with solid tumors.

(2) To establish a recommended dose of lometrexol sodium and gemcitabine HCl given in conjunction with folic acid for study in Phase II trials.

(3) To determine the quantitative and qualitative toxicities of lometrexol sodium and gemcitabine HCl when given in conjunction with folic acid on this schedule.

(4) To determine the plasma concentrations of lometrexol sodium and gemcitabine HCl that are achieved on this schedule, and to relate their pharmacokinetics to toxicity outcome.

(5) To document the antitumor activity of lometrexol sodium and gemcitabine HCl when given in conjunction with folic acid on this schedule.

During each 21-day cycle, patients take folic acid 5 mg orally once daily starting 7 days prior to the start of each cycle and continuing for 7 days after receiving Day 1 of lometrexol sodium and gemcitabine HCl. On Day 1 of each cycle, patients receive an intravenous (IV) bolus dose of lometrexol sodium over approximately 30 to 60 seconds followed by IV infusion of gemcitabine HCl over approximately 30 minutes. Gemcitabine HCl is repeated on Day 8. Treatment cycles are given on an outpatient basis every 21 days. Patients are allowed to receive multiple cycles as long as eligibility and re-treatment criteria continue to be met, toxicity is acceptable, and there is no evidence of disease progression. After the initial dose, adjustments of the dose of lometrexol sodium and gemcitabine are allowed based on individual patient tolerance. Blood and urine sampling is performed during Cycle 1 for lometrexol sodium and gemcitabine HCl pharmacokinetics.

Dosages and Administration

Lometrexol sodium is prepared and administered as described above in Example 2.

Gemcitabine HCl is commercially available from the manufacturer. The drug is reconstituted according to the instructions on the package insert and used as directed. Gemcitabine HCl is soluble in water, slightly soluble in methanol, and insoluble in ethanol and polar organic solvents. Gemcitabine HCl is not a vesicant. The clinical formulation is supplied in a sterile form for intravenous use only. Note: Gemcitabine HCl is administered by intravenous infusion over approximately 30 minutes immediately following the bolus dose of lometrexol sodium on Day 1 of each 21-day cycle. Gemcitabine HCl is administered alone on Day 8.

The dose levels and number of patients at each level are shown in Table 3.

TABLE 3
Dose Levels

| | | Gemcitabine HCl | |
|---|---|---|---|
| | Lometrexol Sodium | (mg/m²) | Number of |
| Dose Level | (mg/m²) Day 1 | Day 1 and Day 8 | Patients |
| −1 | 30 | 800 | 3 to 6 |
| 1 | 50 | 800 | 3 to 6 |
| 2 | 50 | 1000 | 3 to 6 |
| 3 | 75 | 1000 | 3 to 6 |
| 4 | 90 | 1000 | 3 to 6 |
| 5 | 100 | 1250 | 3 to 6 |

### Example 5

### A Phase I Trial of Lometrexol Sodium and Paclitaxel Administered Intravenously Every 21 Days in Conjunction with Oral Folic Acid in Patients with Solid Tumors

This is a Phase I, open-label study of the combination of lometrexol sodium and paclitaxel administered intravenously to patients with locally advanced/metastatic cancer. Paclitaxel is an antimicrotubule agent that promotes the assembly of microtubules from tubulin dimers and stabilizes microtubules by preventing depolymerization. Patients who are eligible for this study have a diagnosis of locally advanced/metastatic solid tumor that has failed conventional treatment or for which no standard therapy is available. A sufficient number of patients are enrolled at up to five dose levels to determine the maximum tolerated dose (MTD) of lometrexol sodium and paclitaxel given in conjunction with folic acid. Approximately 12 to 42 patients are entering the study.

The study objectives are as follows:

(1) To determine the MTD of lometrexol sodium and paclitaxel administered every 21 days in conjunction with folic acid in patients with solid tumors.

(2) To establish a recommended dose of lometrexol sodium and paclitaxel given in conjunction with folic acid for study in Phase II trials.

(3) To determine the quantitative and qualitative toxicities of lometrexol sodium and paclitaxel when given in conjunction with folic acid on this schedule.

(4) To determine the plasma concentrations of lometrexol sodium and paclitaxel that are achieved on this schedule, and to relate their pharmacokinetics to toxicity outcome.

(5) To document the antitumor activity of lometrexol sodium and paclitaxel when given in conjunction with folic acid on this schedule.

During each 21-day cycle, patients take folic acid 5 mg orally once daily starting 7 days prior to the start of each cycle and continuing for 7 days after receiving lometrexol sodium and paclitaxel. On Day 1 of each cycle, patients receive an intravenous bolus dose of lometrexol sodium over approximately 30 to 60 seconds followed by IV infusion of paclitaxel over 3 hours. Treatment is given on an outpatient basis every 21 days. Patients are allowed to receive multiple cycles as long as eligibility and retreatment criteria continue to be met, toxicity is acceptable, and there is no evidence of disease progression. After the initial dose, adjustments of the dose of lometrexol sodium and paclitaxel are allowed based on individual patient tolerance. Blood and urine sampling are performed during Cycle 1 for lometrexol sodium and paclitaxel pharmacokinetics.

Dosages and Administration

Lometrexol sodium is prepared and administered as described above in Example 2.

Paclitaxel is supplied as a nonaqueous solution intended for dilution with a suitable parenteral fluid prior to intravenous infusion. It is commercially available from the manufacturer. The drug should be reconstituted according to the instructions on the package insert and used as directed. Paclitaxel is prepared in glass, polyethylene, or polyolefin containers and administered using IV administration sets (tubing) lined with polyethylene or polyolefin according to the manufacturer's instructions. All patients are premedicated prior to paclitaxel administration in order to prevent severe hypersensitivity reactions. Such premedication consists of either a steroid, an H₂-antagonist, or diphenhydramine. The premedication regimen may be chosen based on institutional guidelines, or the following regimen may be used.

Dexamethasone 20 mg P0, 12 hours and 6 hours before paclitaxel

Diphenhydramine 50 mg IV, 30-60 minutes before paclitaxel

Cimetidine 300 mg IV, 30-60 minutes before paclitaxel or Ranitidine 50 mg IV, 30-60 minutes before paclitaxel or Famotidine 20 mg IV, 30-60 minutes before paclitaxel

Paclitaxel is contraindicated in patients who have a known hypersensitivity to drugs formulated in Cremophor® EL (polyoxyethylated castor oil). The clinical formulation is supplied in a sterile form for intravenous use only. Note: Paclitaxel is administered by intravenous infusion over 3 hours immediately following the bolus dose of lometrexol sodium on Day 1 of each cycle.

The dose levels and number of patients at each level are shown in Table 4.

TABLE 4
Dose Levels

| | Lometrexol Sodium | Paclitaxel (mg/m²) | Number of |
|---|---|---|---|
| Dose Level | (mg/m²) Day 1 | Day 1 | Patients |
| −1 | 30 | 175 | 3 to 6 |
| 1 | 50 | 175 | 3 to 6 |
| 2 | 75 | 175 | 3 to 6 |
| 3 | 100 | 175 | 3 to 6 |
| 4 | 100 | 200 | 3 to 6 |
| 5 | 100 | 225 | 3 to 6 |

### Example 6

### A Phase I Trial of Lometrexol Sodium and Temolozolamide Administered Intravenously in Conjunction with Oral Folic Acid in Patients with Solid Tumors

This is a Phase I, open-label study of the combination of lometrexol sodium and temolozolamide administered orally to patients with locally advanced/metastatic cancer. Patients who are eligible for this study have a diagnosis of locally advanced/metastatic solid tumor that has failed conventional treatment or for which no standard therapy is available. A sufficient number of patients are enrolled at up to seven dose levels to determine the maximum tolerated dose (MTD) of lometrexol sodium and temolozolamide given in conjunction with folic acid. Approximately 12 to 42 patients are entering the study.

The study objectives are as follows:

(1) To determine the MTD of lometrexol sodium and temolozolamide administered in conjunction with folic acid in patients with solid tumors.

(2) To establish a recommended dose of lometrexol sodium and temolozolamide given in conjunction with folic acid for study in Phase II trials. The recommended dose is established in two groups of patients as follows: lightly pretreated and heavily pretreated. Patients who are heavily pretreated are those that fulfill any of the following criteria:

Have previously received radiation to ≧25% of their hematopoietic bone marrow (N.B. Whole pelvic irradiation is ≧25%)

Have received greater than 6 courses of an alkylating agent

Have received greater than 4 courses of carboplatin

Have received greater than 2 courses of mitomycin C or a nitrosourea

Have a primary malignancy with a high propensity for diffuse bone marrow metastases (e.g., prostate cancer, lymphoma)

(3) To determine the quantitative and qualitative toxicities of lometrexol sodium and temolozolamide when given in conjunction with folic acid on this schedule.

(4) To determine the plasma concentrations of lometrexol sodium and temolozolamide that are achieved on this schedule, and to relate their pharmacokinetics to toxicity outcome.

(5) To document the antitumor activity of lometrexol sodium and temolozolamide when given in conjunction with folic acid on this schedule.

During each 28-day cycle, patients take 5 mg folic acid orally once daily starting 7 days prior to the start of each cycle and continuing for 7 days after receiving Day 1 of lometrexol sodium and temolozolamide. On Day 1 of each cycle, patients receive an intravenous (IV) bolus dose of lometrexol sodium over approximately 30 to 60 seconds followed by oral temolozolamide. Oral temolozolamide is repeated on Days 2-5. Treatment cycles are given on an outpatient basis every 28 days. Patients are allowed to receive multiple cycles as long as eligibility and re-treatment criteria continue to be met, toxicity is acceptable, and there is no evidence of disease progression. After the initial dose, adjustments of the dose of lometrexol sodium and temolozolamide are allowed based on individual patient tolerance. Blood and urine sampling are performed during Cycle 1 for lometrexol sodium and temolozolamide pharmacokinetics.

Dosages and Administration

Lometrexol sodium is prepared and administered as described above in Example 2.

Temozolamide is an oral product available in the following capsule sizes: 5, 20, 100 and 250 mg and is commercially available from the manufacturer. Temozolamide capsules are taken orally immediately after the lometrexol sodium.

The dose levels and number of patients at each level are shown in Table 5.

TABLE 5
Dose Levels

| | Lometrexol Sodium | Temozolamide | Number of |
|---|---|---|---|
| Dose Level | (mg/m²) | (mg/m²/day) | Patients |
| −1 | 30 | 100 | 3 to 6 |
| 1 | 50 | 100 | 3 to 6 |
| 2 | 50 | 125 | 3 to 6 |
| 3 | 50 | 150 | 3 to 6 |
| 4 | 75 | 150 | 3 to 6 |
| 5 | 100 | 150 | 3 to 6 |
| 6 | 100 | 200 | 3 to 6 |

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teaching of this invention that certain changes and modifications may be made thereto without departing from the spirit or scope of the appended claims.

What is claimed is:

1. A composition for the treatment of proliferative disorders, comprising:
(i) lometrexol or a pharmaceutically acceptable salt thereof; and
(ii) one or more antiproliferative agents or pharmaceutically acceptable salts thereof.

2. A composition in accordance with claim 1, further comprising folic acid.

3. A composition in accordance with claim 1, wherein said antiproliferative agent is a member selected from the group consisting of alkylating drugs, antimetabolites, microtubule inhibitors, podophyllotoxins, antibiotics, nitrosoureas, hormone therapies, kinase inhibitors, and antiangiogenic agents.

4. A composition in accordance with claim 1, wherein said antiproliferative agent is selected from the group consisting of carboplatin, doxorubicin, gemcitabine HCl, temolozolamide, cyclophosphamide, methotrexate, paclitaxel, etoposide, carmustine, cisplatin, tamoxifen, and interferon.

5. A composition in accordance with claim 3, wherein said kinase inhibitor is selected from the group consisting of tyrphostin AG1478 (4-(3-chloroanilino)-6,7-dimethoxyquinazoline), tyrphostin AG490 (2-cyano-3-(3,4-dihydroxyphenyl)-N-(benzyl)-2-propenamide), indirubin-3′-monoxime, alsterpaullone, genistein, Iressa™ (ZD1839), Gleevec™ (STI-571), SU5416, and Tarceva™ (OSI-774).

6. A method for the treatment of proliferative disorders, comprising administering to a subject in need of such treatment an effective amount of a composition comprising:
(i) lometrexol or a pharmaceutically acceptable salt thereof; and
(ii) one or more antiproliferative agents or pharmaceutically acceptable salts thereof.

7. A method in accordance with claim 6, said composition further comprising folic acid.

8. A method in accordance with claim 6, wherein said proliferative disorder is cancer.

9. A method in accordance with claim 8, wherein said cancer is selected from the group consisting of a solid tumor, a lymphoma, and a leukemia.

10. A method in accordance with claim 9, wherein said solid tumor is selected from the group consisting of ovarian, breast, head and neck, prostate, glioma, colon, stomach, hepatic, renal, chondrocytoma, small cell lung carcinoma, non-small cell lung carcinoma, and melanoma.

11. A method in accordance with claim 6, wherein said proliferative disorder is selected from the group consisting of rheumatoid arthritis, psoriasis, and benign prostatic hyperplasia.

12. A method in accordance with claim 8, wherein said antiproliferative agent is a member selected from the group consisting of alkylating drugs, antimetabolites, microtubule inhibitors, podophyllotoxins, antibiotics, nitrosoureas, hormone therapies, kinase inhibitors, and antiangiogenic agents.

13. A method in accordance with claim 8, wherein said antiproliferative agent is selected from the group consisting of carboplatin, doxorubicin, gemcitabine HCl, temolozolamide, cyclophosphamide, methotrexate, paclitaxel, etoposide, carmustine, cisplatin, tamoxifen, and interferon.

14. A method in accordance with claim 12, wherein said kinase inhibitor is selected from the group consisting of tyrphostin AG1478 (4-(3-chloroanilino)-6,7-dimethoxyquinazoline), tyrphostin AG490 (2-cyano-3-(3,4-dihydroxyphenyl)-N-(benzyl)-2-propenamide), indirubin-3′-monoxime, alsterpaullone, genistein, Iressa™ (ZD1839), Gleevec™ (STI-571), SU5416, and Tarceva™ (OSI-774).

15. A method in accordance with claim 6, wherein said antiproliferative agent is a member selected from the group consisting of alkylating drugs, antimetabolites, microtubule inhibitors, podophyllotoxins, antibiotics, nitrosoureas, hormone therapies, kinase inhibitors, and antiangiogenic agents.

16. A method in accordance with claim 6, wherein said antiproliferative agent is selected from the group consisting of carboplatin, doxorubicin, gemcitabine HCl, temolozolamide, cyclophosphamide, methotrexate, paclitaxel, etoposide, carmustine, cisplatin, tamoxifen, and interferon.

17. A method in accordance with claim 15, wherein said kinase inhibitor is selected from the group consisting of tyrphostin AG1478 (4-(3-chloroanilino)-6,7-dimethoxyquinazoline), tyrphostin AG490 (2-cyano-3-(3,4-dihydroxyphenyl)-N-(benzyl)-2-propenamide), indirubin-3′-monoxime, alsterpaullone, genistein, Iressa™ (ZD1839), Gleevec™ (STI-571), SU5416, and Tarceva™ (OSI-774).

18. A method for the treatment of proliferative disorders, comprising administering to a subject in need of such treatment
(i) an effective first amount of lometrexol or a pharmaceutically acceptable salt thereof; and
(ii) an effective second amount of one or more antiproliferative agents or pharmaceutically acceptable salts thereof.

19. A method in accordance with claim 18, said composition further comprising folic acid.

20. A method in accordance with claim 18, wherein said amount of lometrexol and said amount of antiproliferative agent are administered simultaneously.

21. A method in accordance with claim 18, wherein said amount of lometrexol is administered before said amount of antiproliferative agent.

22. A method in accordance with claim 18, wherein said amount of lometrexol is administered before said amount of antiproliferative agent within a day.

23. A method in accordance with claim 18, wherein said amount of lometrexol is administered before said amount of antiproliferative agent within a week.

24. A method in accordance with claim 18, wherein said amount of antiproliferative agent is administered before said amount of lometrexol.

25. A method in accordance with claim 18, wherein said amount of antiproliferative agent is administered before said amount of lometrexol within a day.

26. A method in accordance with claim 18, wherein said amount of antiproliferative agent is administered before said amount of lometrexol within a week.

27. A method in accordance with claim 18, wherein said proliferative disorder is cancer.

28. A method in accordance with claim 27, wherein said cancer is selected from the group consisting of a solid tumor, a lymphoma, and a leukemia.

29. A method in accordance with claim 28, wherein said solid tumor is selected from the group consisting of ovarian, breast, head and neck, prostate, glioma, colon, stomach, hepatic, renal, chondrocytoma, small cell lung carcinoma, non-small cell lung carcinoma, and melanoma.

30. A method in accordance with claim 18, wherein said proliferative disorder is selected from the group consisting of rheumatoid arthritis, psoriasis, and benign prostatic hyperplasia.

31. A method in accordance with claim 27, wherein said antiproliferative agent is a member selected from the group consisting of alkylating drugs, antimetabolites, microtubule inhibitors, podophyllotoxins, antibiotics, nitrosoureas, hormone therapies, kinase inhibitors, and antiangiogenic agents.

32. A method in accordance with claim 27, wherein said antiproliferative agent is selected from the group consisting of carboplatin, doxorubicin, gemcitabine HCl, temolozolamide, cyclophosphamide, methotrexate, paclitaxel, etoposide, carmustine, cisplatin, tamoxifen, and interferon.

33. A method in accordance with claim 31, wherein said kinase inhibitor is selected from the group consisting of tyrphostin AG1478 (4-(3-chloroanilino)-6,7-dimethoxyquinazoline), tyrphostin AG490 (2-cyano-3-(3,4-dihydroxyphenyl)-N-(benzyl)-2-propenamide), indirubin-3′-monoxime, alsterpaullone, genistein, Iressa™ (ZDI839), Gleeve™ (STI-571), SU5416, and Tarceva™ (OSI-774).

34. A method in accordance with claim 18, wherein said antiproliferative agent is a member selected from the group consisting of alkylating drugs, antimetabolites, microtubule inhibitors, podophyllotoxins, antibiotics, nitrosoureas, hormone therapies, kinase inhibitors, and antiangiogenic agents.

35. A method in accordance with claim 18, wherein said antiproliferative agent is selected from the group consisting of carboplatin, doxorubicin, gemcitabine HCl, temolozolamide, cyclophosphamide, methotrexate, paclitaxel, etoposide, carmustine, cisplatin, tamoxifen, and interferon.

36. A method in accordance with claim 34, wherein said kinase inhibitor is selected from the group consisting of tyrphostin AG1478 (4-(3-chloroanilino)-6,7-dimethoxyquinazoline), tyrphostin AG490 (2-cyano-3-(3,4-dihydroxyphenyl)-N-(benzyl)-2-propenamide), indirubin-3′-monoxime, alsterpaullone, genistein, Iressa™ (ZD1839), Gleevec™ (STI-571), SU5416, and Tarceva™ (OSI-774).
